(19) United States
(12) Patent Application Publication
(10) Pub. No.: US 2016/0192988 A1
(43) Pub. Date: Jul. 7, 2016

(54) Multiple Laser Source System for Portable Laser Therapy Apparatus

(71) Applicant: K-LASER, LLC, Franklin, TN (US)

(72) Inventor: Richard Albright, (US)

(21) Appl. No.: 14/798,106

(22) Filed: Jul. 13, 2015

Related U.S. Application Data
(63) Continuation-in-part of application No. 13/470,976, filed on May 14, 2012, now abandoned.

(51) Int. Cl. A61B 18/22; A61B 18/20; A61N 5/06

(52) U.S. Cl. CPC A61B 18/22; A61N 5/0616; A61B 18/203; A61N 2005/067

(57) ABSTRACT

Provided is a modular multiple laser source system to produce and deliver laser light for laser therapy and/or surgery, the system including a plurality of laser sources of same or different wavelengths, a focusing lens to focus laser light from the plurality of laser sources to a common output, a casing to enclose the laser sources and focusing lens, an output portion provided to the casing to transmit the laser light from one or more of the plurality of laser sources delivered to the common output, and a communication interface provided to the casing to provide electrical communication between components of the system and a controller, wherein the plurality of laser sources are configured to supply the laser light in different wavelengths and/or combinations of wavelengths according to desired medical procedures.

### CROSS-REFERENCE TO RELATED APPLICATIONS

This application is a Continuation-In-Part of U.S. application Ser. No. 13/470,976, filed on May 14, 2012.

### FIELD OF INVENTION

The present general inventive concept relates generally to a portable apparatus to perform laser therapy, and, more particularly, a portable apparatus to apply lasers of different wavelengths and powers to living tissue for therapeutic treatment and/or surgery.

### BACKGROUND

Laser light therapy has become increasingly popular in physiotherapy and surgery applications due to the many benefits available through the application of laser light. Laser light can be used to treat a variety of problems, ranging from relatively mild conditions, such as acne and skin wrinkling, to more complex problems lying deep under the skin, including afflictions of both organs and bones. In many cases, the application of laser therapy may negate the need for conventional pharmaceutical and/or surgical procedures. Different powers, wavelengths, and frequencies are used to target the distinct tissue types associated with the different medical conditions being treated. With the many physical benefits available over the large range of these powers, wavelengths, and frequencies, there exists a need for a device to deliver a large number of different combinations of these values in order to treat a wide variety of conditions. Further, in order for the device to be readily adapted in home and field use as well as medical office and clinical conditions, the device should be readily portable, updatable, and relatively easy to use.

### BRIEF SUMMARY

The present general inventive concept provides a readily portable laser emitting apparatus to conveniently apply laser emissions to a patient for therapeutic treatment and/or surgery. The portable laser emitting apparatus includes one or more laser sources, and may be controlled so as to apply a variety of levels of laser emissions according to different desired therapeutic and surgical procedures.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a portable laser emitting apparatus to be used in physiotherapy and/or surgery, the apparatus including a readily portable housing, one or more laser sources of same or different wavelengths provided in the housing, and a flexible waveguide extending from the housing to transmit laser light from the one or more laser sources to a target area.

The flexible waveguide may include an optical fiber with a core size of approximately 200 um, and an NA of approximately 0.15 to 0.37.

The apparatus may further include a handpiece, provided at a distal end of the flexible waveguide, configured to emit the laser light to the target area.

The handpiece may be configured to be selectively controlled to deliver the laser light in a focus mode or a zoom mode.

The apparatus may further include at least two detachable members to be selectively attached to the handpiece according to selection of the focus mode or the zoom mode.

At least one of the detachable members may be a twist control that is twisted to adjust a contact area of the transmitted laser light in the zoom mode.

A spot size of the laser light delivered in the zoom mode may be adjustable from approximately 1 to 5 cm².

The detachable members may be mechanically or magnetically coupled to the handpiece.

The apparatus may further include a combiner to combine light from the one or more laser sources into the laser light transmitted by the flexible waveguide.

The flexible waveguide may be provided with a pliable metal sheath surrounding the flexible waveguide and control wiring connected to the handpiece.

The one or more laser sources may include a first laser source to transmit laser light having a wavelength of approximately 660 nm at a power up to approximately 100 mW, and a second laser source to transmit laser light having a wavelength of approximately 800 nm in a power range of approximately 0.1 to 12.0 W.

The one or more laser sources may further include a third laser source to transmit laser light having a wavelength of approximately 970 nm at a power range of approximately 0.1 to 12.0 W.

The second and/or third laser source may transmit at a power range of approximately 0.1 to 8.0 W.

The one or more laser sources may further include a fourth laser source to transmit laser light having a wavelength of approximately 905 nm at a power range of approximately 0.1 to 12.0 W.

The frequencies of the laser light may be adjustable between approximately 1 to 20,000 Hz in approximately 1 Hz increments.

Any of the one or more laser sources may be controlled to emit the laser light separately or concurrently.

The apparatus may further include a carrying handle provided to the housing so that the apparatus may be transported by hand by a user.

The apparatus may further include a data storage to store a plurality of predetermined settings of wavelength and power combinations to be emitted from the one or more laser sources, and a controller to control the one or more laser sources to operate according to the predetermined settings.

The apparatus may further include a communication terminal to receive data updates for the controller and/or data storage.

The communication terminal may be a USB port.

The communication terminal may perform wireless communication.

The apparatus may further include a touch screen user interface.

The apparatus may further include a rechargeable battery to supply power to the apparatus.

The one or more laser sources may be light emitting diodes.

The foregoing and/or other aspects and advantages of the present general inventive concept may also be achieved by a modular multiple laser source system to produce and deliver laser light for laser therapy and/or surgery, the system including a plurality of laser sources of same or different wavelengths, a focusing lens to focus laser light from the plurality of laser sources to a common output, a casing to enclose the laser sources and focusing lens, an output portion provided to the casing to transmit the laser light from one or more of the plurality of laser sources delivered to the common output, and a communication interface provided to the casing to provide electrical communication between components of the system and a controller, wherein the plurality of laser sources are configured to supply the laser light in different wavelengths and/or combinations of wavelengths according to desired medical procedures.

The system may further include a steering lens provided in the casing between the laser sources and the focusing lens to redirect the laser light from one or more of the plurality of laser sources to the focusing lens.

The system may further include a power meter inside the casing to measure respective powers of the laser light being transmitted from the laser sources.

The system may further include a partially reflecting lens to reflect at least a portion the laser light from the laser sources to the power meter, and to transmit a remaining portion of the laser light.

The partially reflecting lens may transmit the remaining portion of the laser light directly to the focusing lens.

The system may further include one or more source dedicated lenses provided to one or more of the respective laser sources to provide initial focus of the laser light from the respective laser sources.

The system may further include a connection interface provided to the casing to connect the output portion to a waveguide.

The connection interface may be configured as a screw type connection.

The plurality of laser sources may include a first laser source to transmit laser light having a wavelength of approximately 660 nm at a power up to approximately 100 mW, and a second laser source to transmit laser light having a wavelength of approximately 800 nm in a power range of approximately 0.1 to 12.0 W.

The plurality of laser sources may further include a third laser source to transmit laser light having a wavelength of approximately 970 nm at a power range of approximately 0.1 to 12.0 W.

The second and/or third laser source may transmit at a power range of approximately 0.1 to 8.0 W.

The one or more laser sources may further include a fourth laser source to transmit laser light having a wavelength of approximately 905 nm at a power range of approximately 0.1 to 12.0 W.

The frequencies of the laser light may be adjustable between approximately 1 to 20,000 Hz in approximately 1 Hz increments.

Any of the one or more laser sources may be controlled to emit the laser light separately or concurrently.

The plurality of laser sources may be light emitting diodes.

The foregoing and/or other aspects and advantages of the present general inventive concept may also be achieved by a portable laser emitting apparatus to be used in physiotherapy and/or surgery, the apparatus including a readily portable housing, a modular multiple laser source system provided in the housing to produce and deliver laser light, the system including a plurality of laser sources of same or different wavelengths, a focusing lens to focus laser light from the plurality of laser sources to a common output, a casing to enclose the laser sources and focusing lens, an output portion provided to the casing to transmit the laser light from one or more of the plurality of laser sources delivered to the common output, and a communication interface provided to the casing to provide electrical communication between components of the system and a controller, and a flexible waveguide extending from the housing to transmit laser light from the one or more laser sources to a target area.

The flexible waveguide may include an optical fiber with a core size of approximately 400 um.

The apparatus may further include a handpiece, provided at a distal end of the flexible waveguide, configured to emit the laser light to the target area.

The flexible waveguide may be provided with a pliable metal sheath surrounding the flexible waveguide and control wiring connected to the handpiece.

The plurality of laser sources may include a first laser source to transmit laser light having a wavelength of approximately 660 nm at a power up to approximately 100 mW, a second laser source to transmit laser light having a wavelength of approximately 800 nm in a power range of approximately 0.1 to 12.0 W, a third laser source to transmit laser light having a wavelength of approximately 970 nm at a power range of approximately 0.1 to 12.0 W, a fourth laser source to transmit laser light having a wavelength of approximately 905 nm at a power range of approximately 0.1 to 12.0 W.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a portable laser emitting apparatus according to an example embodiment of the present general inventive concept;

FIG. 2 is a schematic illustration of some of the components of the portable laser emitting apparatus of FIG. 1, according to an example embodiment of the present general inventive concept;

FIG. 3 illustrates example elements and functions of the handpiece of the portable laser emitting apparatus according to an example of the present general inventive concept;

FIG. 4 is a graph illustrating the application of the laser light in the continuous wave mode;

FIG. 5 is a graph illustrating the application of the laser light in the frequency modulated, or pulsed emission, mode;

FIG. 6 is a graph illustrating the application of the laser light in the intense super pulse mode;

FIG. 7 illustrates a portable laser emitting apparatus according to another example embodiment of the present general inventive concept;

FIG. 8 illustrates a self-contained multiple laser source system according to an example embodiment of the present general inventive concept; and

FIG. 9 illustrates an external view of the self-contained multiple laser source system of FIG. 8 according to an example embodiment of the present general inventive concept.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Various embodiments of the present general inventive concept, as described herein, provide a laser emitting apparatus that is lightweight and readily portable. In other words, the apparatus is designed so as to be easily transported from room to room, or between different locations in the same room, and therefore may be conveniently used in a home or office environment. The portable laser emitting apparatus may be used for physiotherapy and/or surgery on human and/or animal subjects, and may also have application on non-living subjects. Such various applications will be evident to one skilled in the art. In most of the various example embodiments described herein, a target area of a human subject is discussed, and the human subject may be referred to as the patient. However, it is understood that the use of laser light emitted from the portable laser emitting apparatus is not limited to such an application.

To further increase the ease of use of the portable laser emitting apparatus, according to various embodiments of the present general inventive concept, one or more laser sources of same or different wavelengths are provided in a housing of the portable laser emitting apparatus, and the laser light emitted from these laser sources is transmitted through a flexible waveguide to then be applied to a target area of the patient.

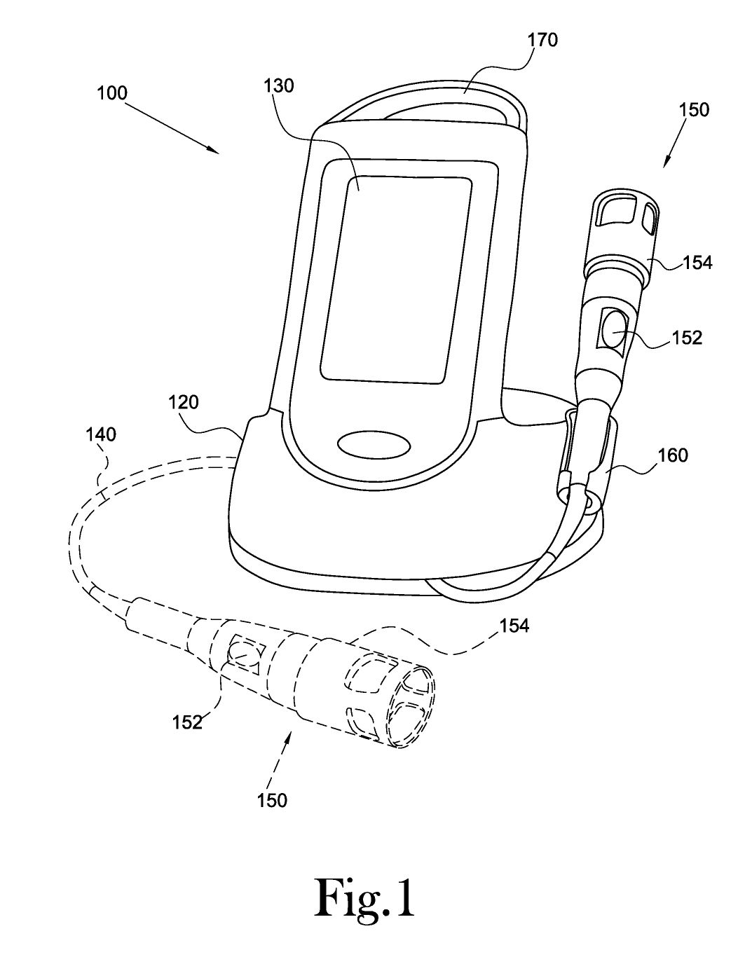

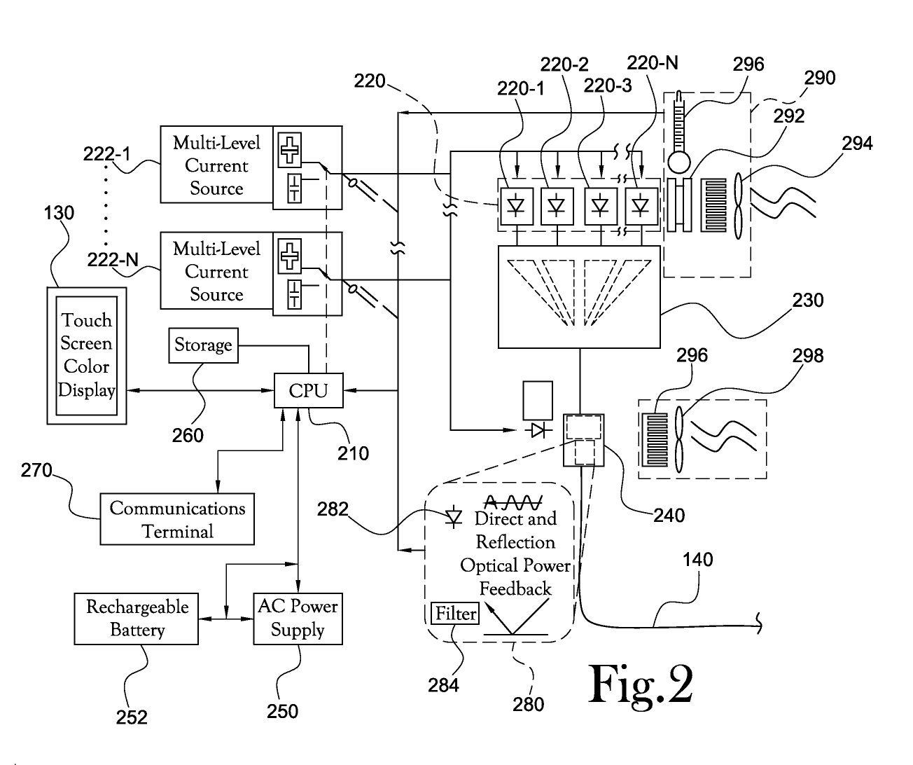

FIG. 1 illustrates a portable laser emitting apparatus according to an example embodiment of the present general inventive concept. Referring to FIG. 1, this example embodiment of the portable laser emitting apparatus 100 includes housing 120 to enclose and protect many of the components of the laser emitting apparatus 100, a touch screen display 130 to provide user interaction with the portable laser emitting apparatus, a flexible waveguide 140 to transmit laser light away from light sources provided in the housing 120 (discussed in more detail in the description of FIG. 2), and a handpiece 150 provided at a distal end of the flexible waveguide 140 to emit the laser light delivered through the flexible waveguide 140 to a target area of a patient. The example embodiment illustrated in FIG. 1 also includes a handpiece docking portion 160 which my secure the handpiece 140 when the portable laser emitting apparatus 100 is not in use, as well as a handle 170 to increase the convenience enjoyed by a user when transporting the device. To further increase the user's convenience regarding portability, in some example embodiments the portable laser emitting apparatus 100 is constructed of such lightweight material(s) as to weigh less than or equal to approximately 3.5 pounds. In other words, the portable laser emitting apparatus 100 is fabricated so as to be readily carried by a user, such as by hand, to easily move from room to room without aid from other people or mechanical aid.

It will be understood by one skilled in the art that various example embodiments of the present general inventive concept may omit various elements described in regard to the illustrated example embodiments, and that various other elements may be added. Further, the configurations of the portable laser emitting apparatus 100 are merely example configurations, and may be altered according to the various design and/or use preferences. Various components, such as the touch screen display 130, may be integrated along with the housing 120 of the portable laser emitting apparatus 100, or may be modular in order to be readily removed in the event that repair or replacement is desired.

Although not illustrated in FIG. 1, the portable laser emitting apparatus 100 may be provided with a power connection, such as an electrical cord, to be connected with an AC power source, as well as a rechargeable battery which may be provided inside or outside of the housing 120. In various example embodiments, the rechargeable battery may be removable from the portable laser emitting apparatus 100 to be recharged, and in other various example embodiments the rechargeable battery may be recharged solely by power supplied by the AC power source. In other various example embodiments of the present general inventive concept, the portable laser emitting apparatus may be equipped to receive power through wireless power transmission.

Although a touch screen display 130 is illustrated in FIG. 1, it will be understood by one skilled in the art that various other user interfaces may be employed in the portable laser emitting apparatus. For example, a conventional display and keypad combination may be substituted for the touch screen display 130. In various example embodiments, the touch screen display 130 can display images in 16 million color combinations.

The flexible waveguide 140 may be an optical cable, such as a single emitting fiber with a core size of approximately 200 um, and an NA of approximately 0.15 to 0.37. However, the waveguide 140 is not limited to an optical cable, nor an optical cable with these example attributes.

The flexible waveguide 140 may be provided with a pliable metal sheath surrounding the flexible waveguide to protect the flexible waveguide 140, and to house control wiring connected to the handpiece 150.

The example handpiece 150 illustrated in FIG. 1 is provided with at least one switch 152 to enable a user to switch between various laser application modes, and/or to start and stop emission of laser light from the handpiece 150, which will be discussed in more detail later in this description. The example handpiece 150 is also provided with a twist control 154 to enable a user to adjust the focus area of the laser application in a zoom mode, which will also be discussed in more detail later in this description. It will be understood that the at least one switch 152 and twist control 154 are merely example embodiments of the controls provided to the handpiece 150 to aid the operation by a user. Other types of controls may be added or substituted on the handpiece, or such controls may be located on the housing 120 of the portable laser emitting apparatus 100. For example, control of the laser application modes and focus areas may be controlled through the touch screen display 130 illustrated in FIG. 1.

FIG. 2 is a schematic illustration of some of the components of the portable laser emitting apparatus 100 of FIG. 1, according to an example embodiment of the present general inventive concept. According to the example embodiment illustrated in FIG. 2, the portable laser emitting apparatus 100 is provided with a CPU 210 to control the functions of the various provided components. The CPU 210 is in electrical communication with the touch screen display 130, such that commands entered by the user are then carried out by the CPU 210, and results associated with the commands, as well as other various feedback, are displayed to the user.

One or more laser sources 220 may be provided inside the housing 120. In the example embodiment illustrated in FIG. 2, multiple laser sources 220-1 through 220-N are provided inside the housing 120. However, it will be understood that any number of laser sources 220 may be provided, including a single laser source 220. The one or more laser sources 220 may have the same or different output wavelengths and/or powers. In various example embodiments, the one or more laser sources 220 may be light emitting diodes, and may be controlled to operate separately or concurrently.

In an example embodiment such as the one illustrated in FIG. 2, in which multiple laser sources 220 are provided in the housing 120, an optical combiner 230 may be provided to combine the outputs of the multiple laser sources 220 before the outputs enter the flexible waveguide 140.

The portable laser emitting apparatus may also be provided with an optical coupler 240 coupled at some point to the flexible waveguide 140. An additional laser source 242 may be provided to the optical coupler 240, and may have a wavelength between approximately 400 nm and 700 nm. The additional laser source 242 may be collimated at the emission point of the optical coupler 240, and aligned with the flexible waveguide 140. In various example embodiments, the additional laser source 242 may emit light to serve as a guiding beam to indicate an approximate point at which the energy is delivered on the target, i.e., on the tissue, and thus may function as a pointing ray, and/or in various example embodiments the additional laser source 242 may function as an additional therapeutic laser source having a biostimulating effect that may be collimated at the emission point of the optical coupler 240 and aligned with the flexible waveguide 140.

In the example embodiment illustrated in FIG. 2, the output of each of the one or more laser sources 220 are respectively controlled by corresponding one or more multi-level current sources 222-1 through 222-N. The multi-level current sources 222-1 through 222-N are controlled by the CPU 210 to adjust the output of the one or more laser sources 220 to the respective desired levels. The multi-level current sources 222-1 through 222-N may maintain the light output of the respective one or more laser sources 220 constant within 1%.

As previously discussed in regard to FIG. 1, power may be provided to the portable laser emitting apparatus 100 through an AC power supply 250, either connected to a source such as a wall adapter or received wirelessly, or through a rechargeable battery 252. According to various example embodiments, the rechargeable battery may be an internally or externally provided LiFePO4 battery, which may be removable for charging, or may be fixed and charged through the AC power supply 250.

In the example embodiment illustrated in FIG. 2, a data storage 260 is provided in electrical communication with the CPU 210 in order to store a number of preset levels to control output of the one or more laser sources 220. According an example embodiment, a total of 55 different output levels may be stored in the data storage 260, and may be easily selected by the user rather than fine-tuning the outputs of the one or more laser sources 220 to desired levels. The data storage 260 may also be used to store a control system used by the CPU 210 to control the operations of the portable laser emitting apparatus 100. Any of a number of data storage devices, such as, for example, a flash memory device, may be provided as the data storage 260.

A communication terminal 270 is also provided to the example embodiment illustrated in FIG. 2. The communication terminal may be used to provide any system and/or memory updates to the portable laser emitting apparatus 100. For example, the control system or output level presets may be updated or expanded. The communication terminal may be, for example, a USB port used to connect the portable laser emitting apparatus 100 to a computer or other electronic device to download the update data. As another example, the communication terminal may be a wireless connector, such as a Wi-Fi connector, to receive the update data in a wireless fashion.

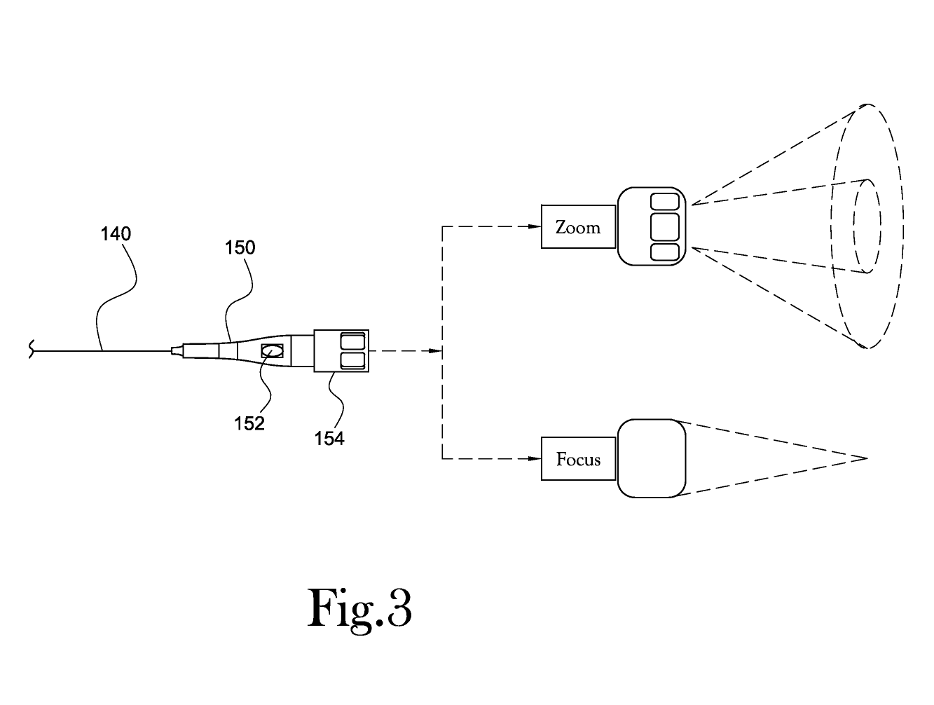

FIG. 3 illustrates example elements and functions of the handpiece 150 of the portable laser emitting apparatus 100 according to an example of the present general inventive concept. As previously described, the handpiece 150 is connected to the portable laser emitting apparatus 100 by the flexible waveguide 140. As also previously described, the handpiece 150 may be provided with at least one switch 152 to enable a user to switch between various laser application modes, and/or to start and stop emission of laser light from the handpiece 150. For example, the at least one switch 152 may be used to switch the handpiece 150 between an ON and OFF state. The at least one switch 152 may also be used to switch between focus and zoom modes of delivery of the laser light. The example handpiece 150 may also be provided with a twist control 154 to enable a user to adjust the focus area of the laser application in a zoom mode.

As illustrated in FIG. 3, the focus mode may be used to concentrate the laser light in a much smaller area of the patient, while the zoom mode may be used to spread the laser light to a comparatively larger area. In one example embodiment, the total area to which the laser light may be applied, according to the distance from the handpiece 150 to the target area, may be approximately 1.0 to 5.0 cm². The focus and zoom modes may be enable by detachable attachments to the handpiece 150, and may be mechanically or magnetically attached to the handpiece 150.

The laser emission may be applied through the handpiece 150 to the patient in several modes according to various example embodiments of the present general inventive concept. For example, according to one example embodiment, the laser light may be applied to the patient in a continuous wave mode, a frequency modulated mode, or an intense super pulse mode. These three example modes are illustrated in FIGS. 4-6.

FIG. 4 is a graph illustrating the application of the laser light in the continuous wave mode. In the continuous wave mode, the laser emission time overlaps the laser activation time, and the power of the laser emission is approximately constant.

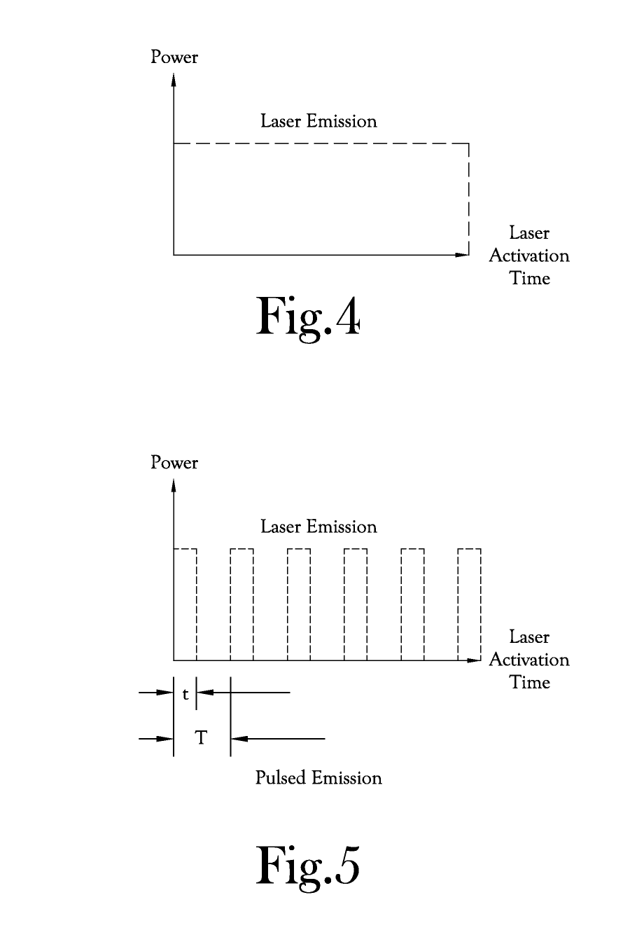

FIG. 5 is a graph illustrating the application of the laser light in the frequency modulated, or pulsed emission, mode. In the pulsed emission mode, the laser light is only emitted in periodic pulses during the laser activation time. The laser light is emitted for a time t during each period T of the laser activation time. The duty cycle is defined as the ratio t/T, or:

Duty Cycle=t/T×100%.

The frequency of pulsed emission is the reciprocal of “T”, or:

Frequency of pulsed laser emission (Hz)=1/T.

For example, if the pulsed laser period is 1 millisecond, the frequency of pulsed laser emission would be 1 kHz.

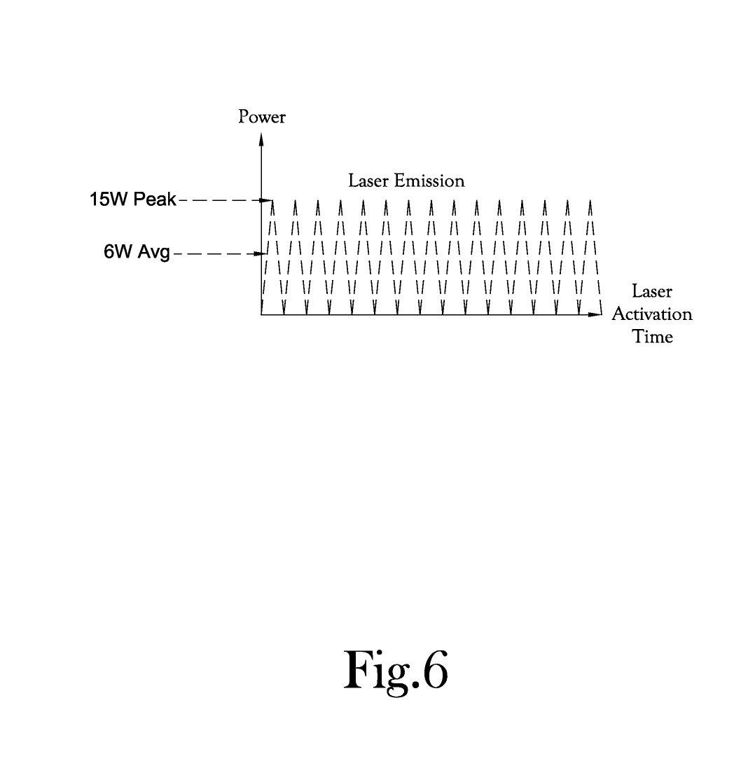

FIG. 6 is a graph illustrating the application of the laser light in the intense super pulse mode. In contrast to the continuous wave and pulsed emission modes, in which the power of the laser light is relatively constant during emission, in the intense super pulse mode the power of the laser emission is periodically and linearly adjusted between a low power and a peak power. In the example intense super pulse mode illustrated in FIG. 6, the peak power of the super pulse is approximately 15 W, while the average power output over time is approximately 6 W.

As previously discussed in regard to FIG. 2, one or more laser sources 220, having the same or different wavelengths, may be provided to the portable laser emitting apparatus 100. The one or more laser sources 220 are provided with current by the respective multi-level current sources 221 to respectively adjust the duration, intensity, and power output of the one or more laser sources 220. A few example embodiments of these one or more laser sources 220, configured and operated accordingly to better treat various medical conditions, will now be described.

In one example embodiment of the present general inventive concept, the portable laser emitting apparatus is provided with a first laser source 220-1 and a second laser source 220-2. The first laser source 220-1 may have a wavelength of 660 nm, with a power output of up to 100 mW, which is known to have stimulatory effects in superficial dermatological conditions such as open wounds, diabetic ulcers and infections. The second laser source 220-2 may have a wavelength of 800 nm, with a power output ranging from 0.1 to 8 or 12 Watts. This second laser source 220-2 is in the NIR range and is centered at the peak of cytochrome c oxidase's absorption.

Another example embodiment may include the first and second laser sources 220-1,220-2 described above, along with a third laser source 220-3, which may have a wavelength of 970 nm, with a power output ranging from 0.1 to 8 or 12 Watts. This wavelength is also in the NIR range and is centered at the peak of water's absorption. This third laser source 220-3 may create thermal gradients on the cellular level along which blood would more readily flow.

Yet another example embodiment may include the first through third laser sources 220-1,220-2,220-3 described above, along with a fourth laser source 220-4, which may have a wavelength of 905 nm, with a power output ranging from 0.1 to 8 or 12 Watts. This fourth laser source 220-4 sits at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. In other words, the present general inventive concept is not limited to the several examples described above.

According to various example embodiments, normal frequency modulated operation, such as that illustrated in FIG. 5, may provide a frequency range adjustable from 1 to 20,000 Hz with an average power ranging from 0.1 to 6 Watts. In various example embodiments of the intense super pulse mode, the frequency range may remain adjustable from 1 to 20,000 Hz, but the peak power may be 21 Watts, with the average power being adjustable from 0.1 to 8 Watts.

The example embodiment illustrated in FIG. 2 also includes various safety features. One such feature is the luminous emission feedback system 280. Included in this luminous emission feedback system 280 is a photodiode 282 and a special direct and reflection optical power system filter 284. Through this filter 284, the photodiode 282 can detect the luminous emissions coming from the irradiated tissues of the patient, which can occur at different wavelengths. These luminous emissions are proportional to the absorption and temperature increase in the patient caused by the emission of laser light from the handpiece 150. The data from the luminous emission feedback system 280 is transmitted to the CPU 210 for analysis and system control. In particular, the luminous emission feedback system 280 insures the efficacy of the optical treatment and maintains the safety level high and constant. Further, this luminous emission feedback system 280 allows an automatic check on the intensity of the laser light delivered to the tissue to optimize the therapeutic effects while guaranteeing maximum safety. Still further, this luminous emission feedback system 280 can gather information for eventual diagnostic purposes.

Another example safety feature in the example embodiment illustrated in FIG. 2 is the use of a cooling system 290 for the one or more laser sources 220. For example, a peltier cooler 292 and fan 294 may be provided to the one or more laser sources 220. Both the peltier cooler 292 and fan 294 may be electrically connected to and under the control of the CPU 210. A temperature gage 296 may be mounted on the one or more laser sources 220 and connected to the CPU 210 so that the CPU 210 can monitor and control the temperature of the one or more laser sources 220 through the control of the peltier cooler 292 and fan 294. This is merely one example through which a safe temperature of the one or more laser sources 220 may be maintained, and is not intended to limit the ways that one can control and maintain proper temperature of the laser sources. One skilled in the art would be aware of other methods for controlling the laser sources in a safe operating temperature range.

A further safety feature illustrated in the example of FIG. 2 is through the providing of a heat sink 296 and a fan 298 on the optical coupler 240, which ensures that the optical coupler 240 remains at a safe operating temperature.

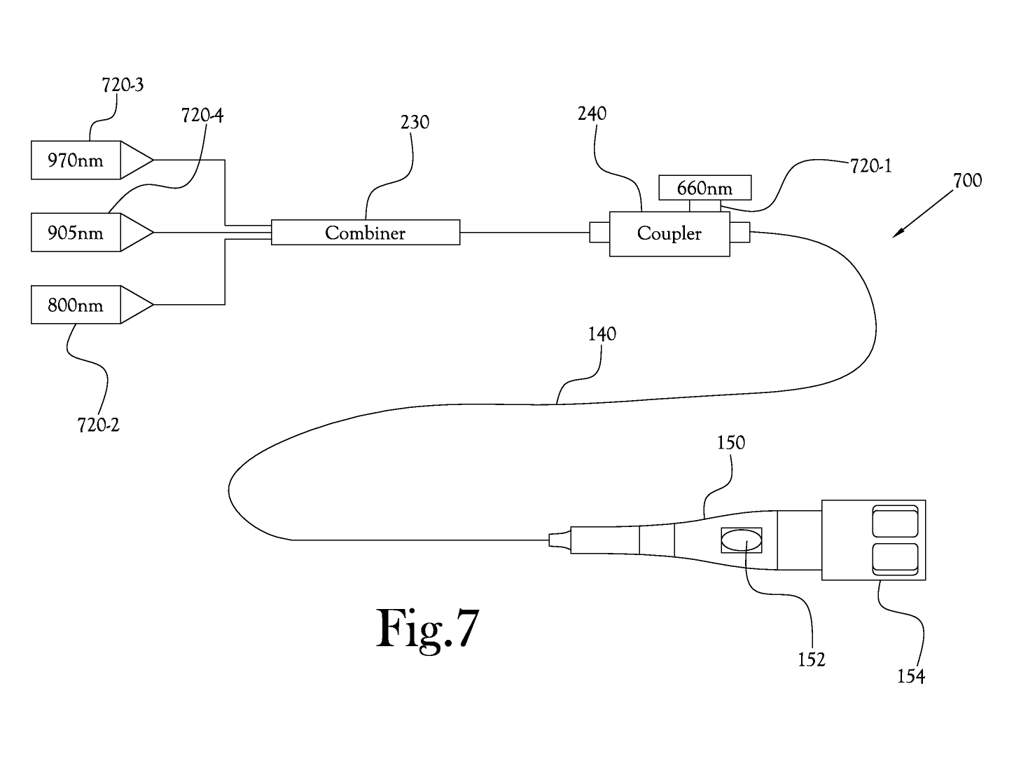

As previously described, the example embodiment of the laser emitting apparatus illustrated in FIGS. 1-2 includes a plurality of lasers 220 having outputs that are combined by a combiner 230 before the laser outputs enter the flexible waveguide 140. As also previously described in regard to the example embodiment illustrated in FIGS. 1-2, an optical coupler 240 may be provided to the flexible waveguide 140, through which an additional laser source may be collimated at the emission point of the optical coupler 240, and aligned with the flexible waveguide 140. FIG. 7 illustrates a portable laser emitting apparatus according to another example embodiment of the present general inventive concept. According to the example embodiment of the laser emitting apparatus 700 configured as illustrated in FIG. 7, a first laser source 720-1, having a wavelength of 660 nm, is provided to the optical coupler 240 to serve as a guiding beam and/or a therapeutic laser source having stimulatory effects in superficial dermatological conditions such as open wounds, diabetic ulcers and infections. A plurality of laser sources 720-2,720-3,720-4 are provided to the laser emitting apparatus 700, the outputs of which are combined by the combiner 230 before the laser outputs enter the flexible waveguide 140 and go on to pass through the coupler 240.

The second laser source 720-2 may have a wavelength of 800 nm, with a power output ranging from 0.1 to 8 or 12 Watts, being in the NIR range, and centered at the peak of cytochrome c oxidase's absorption. The third laser source 720-3 may have a wavelength of 970 nm, with a power output ranging from 0.1 to 8 or 12 Watts, also being in the NIR range, and centered at the peak of water's absorption. This third laser source 720-3 may create thermal gradients on the cellular level along which blood would more readily flow. The fourth laser source 720-4 may have a wavelength of 905 nm, with a power output ranging from 0.1 to 8 or 12 Watts, sitting at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. In other words, the present general inventive concept is not limited to the several examples described above.

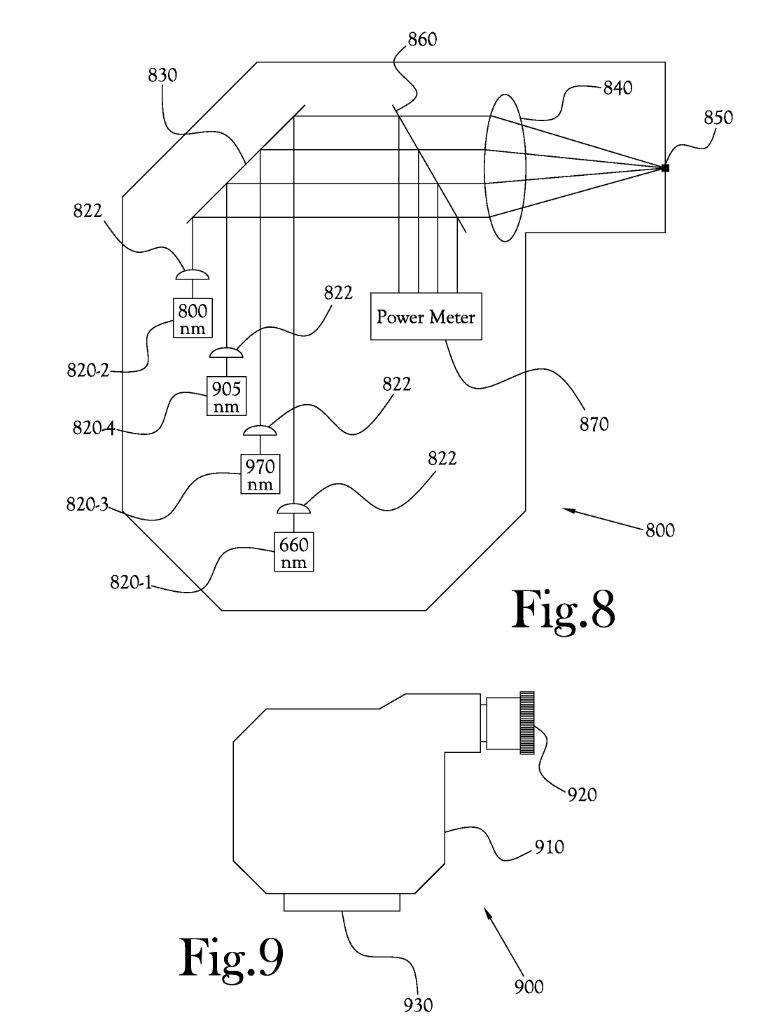

In various example embodiments of the present general inventive concept, the portable laser emitting apparatus may be provided with one or more components that allow the apparatus to be produced and function without the previously described combiner and coupler, which in some configurations may provide greater and more efficient laser outputs, and/or a more compact portable laser emitting apparatus. FIG. 8 illustrates a self-contained multiple laser source system according to an example embodiment of the present general inventive concept. Use of such a self-contained or modular system allows the portable laser emitting apparatus of the present general inventive concept to be manufactured and operate without the previously described combiner and coupler.

The example self-contained multiple laser source system 800 configured as illustrated in FIG. 8 includes a plurality of laser sources 820-1,820-2,820-3,820-4. It is understood that various other example embodiments of the present general inventive concept may include a smaller or larger number of laser sources, and the quantity illustrated in FIG. 8 and discussed herein is merely one example embodiment. For ease of description, the laser sources illustrated in FIG. 8 may have the same values as those discussed in other example embodiments described above, but the laser sources are not limited to such values. In other words, the first laser source 820-1 may have a wavelength of 660 nm at a power up to approximately 100 mW, and may serve as a guiding beam and/or a therapeutic laser source having stimulatory effects in superficial dermatological conditions such as open wounds, diabetic ulcers and infections. The second laser source 820-2 may have a wavelength of 800 nm, with a power output ranging from 0.1 to 8 or 12 Watts, being in the NIR range, and centered at the peak of cytochrome c oxidase's absorption. The third laser source 820-3 may have a wavelength of 970 nm, with a power output ranging from 0.1 to 8 or 12 Watts, also being in the NIR range, and centered at the peak of water's absorption. This third laser source 820-3 may create thermal gradients on the cellular level along which blood would more readily flow. The fourth laser source 820-4 may have a wavelength of 905 nm, with a power output ranging from 0.1 to 8 or 12 Watts, sitting at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. In other words, the present general inventive concept is not limited to the several examples described above.

According to various example embodiments of the present general inventive concept, each of the laser sources 820-1,820-2,820-3,820-4 may be respectively provided with a source dedicated lens 822 to focus the laser output leaving the respective laser sources. In example embodiments, the laser sources may be light emitting diodes, and may be controlled to operate separately or concurrently. A steering lens 830 may be provided to steer the output from the one or more laser sources 820-1,820-2,820-3,820-4 toward a focusing lens 840, which focuses the one or more laser outputs to a common output such as a waveguide interface 850. In other words, when one or more of the laser sources 820-1,820-2,820-3,820-4 is controlled to emit a laser in the example embodiment illustrated in FIG. 8, the one or more laser outputs are transmitted in the direction of the steering lens 830, which reflects the one or more laser outputs to the focusing lens 840, at which point the one or more laser outputs are focused on the waveguide interface 850 to move down a waveguide such as the optical fiber of the previously described flexible waveguide 140. In various example embodiments, such as the example embodiment illustrated in FIG. 8, a partially reflecting lens 860 may be provided in the path of the one or more laser outputs to reflect a portion of the laser outputs to a power meter 870 provided in the self-contained multiple laser source system 800, while transmitting the remaining non-reflected portion of the respective laser outputs. The power meter 870 of the example embodiment illustrated in FIG. 8 may be provided in lieu of the luminous emission feedback system 280 illustrated in FIG. 2. The power measurement data from the power meter 870 may be transmitted to the previously described CPU 210 for analysis and system control, and may allow an automatic check on the intensity of the laser light delivered from the one or more laser sources to optimize the therapeutic effects while guaranteeing maximum safety. Thus, as illustrated in FIG. 8, the self-contained multiple laser source system 800 may be provided with a focusing lens that performs a function otherwise provided by the combiner 230 of the example embodiment illustrated in FIG. 2, and the power meter 870 may perform at least some of the function otherwise provided by the luminous emission feedback system 280 of FIG. 2. Further, as the laser source provided to the coupler 240 of FIG. 2 is located inside of the self-contained multiple laser source system 800 of FIG. 8, the device illustrated in FIG. 8 allows an example embodiment of the portable laser emitting apparatus to be configured without the combiner 230 and/or coupler 240 illustrated in FIG. 2. Such a configuration may allow a portable laser emitting apparatus to have a greater and more efficient laser output.

FIG. 9 illustrates an external view of the self-contained multiple laser source system of FIG. 8 according to an example embodiment of the present general inventive concept. The example self-contained multiple laser source system 900 of FIG. 9 is provided with a casing 910 to enclose and protect the components illustrated in FIG. 8, as well as additional components and wiring which would be understood by one skilled in the art. In an example embodiment, the size of the casing may be approximately 1.5″ tall and 1.25″ wide. The casing 910 may be provided with a connection interface 920 to connect the system 900 to a waveguide such as the flexible waveguide 140 through the waveguide interface 850. For example, the connection interface 920 may be embodied as a rotatable female connection to receive a male connection from an end of the flexible waveguide 140 to be secured with a screw type configuration, such as with a typical coaxial cable. However, it will be noted by those skilled in the art that any of a number of different connection interfaces may be provided to secure the flexible waveguide 140 to the self-contained multiple laser source system 900. In various example embodiments of a laser emitting apparatus that includes the system 900, the optical fiber provided inside the flexible waveguide 140 may have a core size of approximately 400 um. The casing 910 may be provided a communication interface 930 to provide a wired connection to a controller or control component such as the CPU 210 of FIG. 2. With such a configuration, the laser sources 822-1,822-2,822-3,822-4 may be controlled by the CPU 210 to adjust the output of the one or more laser sources to the respective desired levels. The laser sources 822-1,822-2,822-3,822-4 may maintain the output of the respective one or more laser sources constant within 1%.

According to various embodiments of the present general inventive concept, a portable laser emitting apparatus is provided which may be readily moved from location to location in a home or office, either in the same room or different rooms. The portable laser emitting apparatus is constructed such that it is lightweight enough to be easily moved by hand, and through a provided waveguide conveniently transmit laser light from one or more laser sources to a patient.

According to various embodiments of the present general inventive concept, a modular multiple laser source system configured to produce and deliver laser light for laser therapy and/or surgery may include a plurality of laser sources of same or different wavelengths, a focusing lens to focus laser light from the plurality of laser sources to a common output, a casing to enclose the laser sources and focusing lens, an output portion provided to the casing to transmit the laser light from one or more of the plurality of laser sources delivered to the common output, and a communication interface provided to the casing to provide electrical communication between components of the system and a controller, wherein the plurality of laser sources are configured to supply the laser light in different wavelengths and/or combinations of wavelengths according to desired medical procedures. The system may further include a steering lens provided in the casing between the laser sources and the focusing lens to redirect the laser light from one or more of the plurality of laser sources to the focusing lens. The system may further include a power meter inside the casing to measure respective powers of the laser light being transmitted from the laser sources. The system may further include a partially reflecting lens to reflect at least a portion the laser light from the laser sources to the power meter, and to transmit a remaining portion of the laser light. The partially reflecting lens may transmit the remaining portion of the laser light directly to the focusing lens. The system may further include one or more source dedicated lenses provided to one or more of the respective laser sources to provide initial focus of the laser light from the respective laser sources. The system may further include a connection interface provided to the casing to connect the output portion to a waveguide. The connection interface may be configured as a screw type connection. The plurality of laser sources may include a first laser source to transmit laser light having a wavelength of approximately 660 nm at a power up to approximately 100 mW, and a second laser source to transmit laser light having a wavelength of approximately 800 nm in a power range of approximately 0.1 to 12.0 W. The plurality of laser sources may further include a third laser source to transmit laser light having a wavelength of approximately 970 nm at a power range of approximately 0.1 to 12.0 W. The second and/or third laser source may transmit at a power range of approximately 0.1 to 8.0 W. The one or more laser sources may further include a fourth laser source to transmit laser light having a wavelength of approximately 905 nm at a power range of approximately 0.1 to 12.0 W. The frequencies of the laser light may be adjustable between approximately 1 to 20,000 Hz in approximately 1 Hz increments. Any of the one or more laser sources may be controlled to emit the laser light separately or concurrently. The plurality of laser sources may be light emitting diodes.

According to various embodiments of the present general inventive concept, a portable laser emitting apparatus to be used in physiotherapy and/or surgery may include a readily portable housing, a modular multiple laser source system provided in the housing to produce and deliver laser light, the system including a plurality of laser sources of same or different wavelengths, a focusing lens to focus laser light from the plurality of laser sources to a common output, a casing to enclose the laser sources and focusing lens, an output portion provided to the casing to transmit the laser light from one or more of the plurality of laser sources delivered to the common output, and a communication interface provided to the casing to provide electrical communication between components of the system and a controller, and a flexible waveguide extending from the housing to transmit laser light from the one or more laser sources to a target area. The flexible waveguide may include an optical fiber with a core size of approximately 400 um. The apparatus may further include a handpiece, provided at a distal end of the flexible waveguide, configured to emit the laser light to the target area. The flexible waveguide may be provided with a pliable metal sheath surrounding the flexible waveguide and control wiring connected to the handpiece. The plurality of laser sources may include a first laser source to transmit laser light having a wavelength of approximately 660 nm at a power up to approximately 100 mW, a second laser source to transmit laser light having a wavelength of approximately 800 nm in a power range of approximately 0.1 to 12.0 W, a third laser source to transmit laser light having a wavelength of approximately 970 nm at a power range of approximately 0.1 to 12.0 W, a fourth laser source to transmit laser light having a wavelength of approximately 905 nm at a power range of approximately 0.1 to 12.0 W.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A modular multiple laser source system to produce and deliver laser light for laser therapy and/or surgery, the system comprising:
a plurality of laser sources of same or different wavelengths;
a focusing lens to focus laser light from the plurality of laser sources to a common output;
a casing to enclose the laser sources and focusing lens;
an output portion provided to the casing to transmit the laser light from one or more of the plurality of laser sources delivered to the common output; and
a communication interface provided to the casing to provide electrical communication between components of the system and a controller;
wherein the plurality of laser sources are configured to supply the laser light in different wavelengths and/or combinations of wavelengths according to desired medical procedures.

2. The system of claim 1, further comprising a steering lens provided in the casing between the laser sources and the focusing lens to redirect the laser light from one or more of the plurality of laser sources to the focusing lens.

3. The system of claim 1, further comprising a power meter inside the casing to measure respective powers of the laser light being transmitted from the laser sources.

4. The system of claim 3, further comprising a partially reflecting lens to reflect at least a portion the laser light from the laser sources to the power meter, and to transmit a remaining portion of the laser light.

5. The system of claim 4, wherein the partially reflecting lens transmits the remaining portion of the laser light directly to the focusing lens.

6. The system of claim 1, further comprising one or more source dedicated lenses provided to one or more of the respective laser sources to provide initial focus of the laser light from the respective laser sources.

7. The system of claim 1, further comprising a connection interface provided to the casing to connect the output portion to a waveguide.

8. The system of claim 7, wherein the connection interface is configured as a screw type connection.

9. The system of claim 1, wherein the plurality of laser sources comprise:
a first laser source to transmit laser light having a wavelength of approximately 660 nm at a power up to approximately 100 mW; and
a second laser source to transmit laser light having a wavelength of approximately 800 nm in a power range of approximately 0.1 to 12.0 W.

10. The system of claim 9, wherein the plurality of laser sources further comprise a third laser source to transmit laser light having a wavelength of approximately 970 nm at a power range of approximately 0.1 to 12.0 W.

11. The system of claim 10, wherein the second and/or third laser source transmits at a power range of approximately 0.1 to 8.0 W.

12. The system of claim 10, wherein the one or more laser sources further comprise a fourth laser source to transmit laser light having a wavelength of approximately 905 nm at a power range of approximately 0.1 to 12.0 W.

13. The system of claim 9, wherein frequencies of the laser light are adjustable between approximately 1 to 20,000 Hz in approximately 1 Hz increments.

14. The system of claim 1, wherein any of the one or more laser sources are controlled to emit the laser light separately or concurrently.

15. The system of claim 1, wherein the plurality of laser sources are light emitting diodes.

16. A portable laser emitting apparatus to be used in physiotherapy and/or surgery, the apparatus comprising:
a readily portable housing;
a modular multiple laser source system provided in the housing to produce and deliver laser light, the system comprising:
a plurality of laser sources of same or different wavelengths,
a focusing lens to focus laser light from the plurality of laser sources to a common output,
a casing to enclose the laser sources and focusing lens,
an output portion provided to the casing to transmit the laser light from one or more of the plurality of laser sources delivered to the common output, and
a communication interface provided to the casing to provide electrical communication between components of the system and a controller; and
a flexible waveguide extending from the housing to transmit laser light from the one or more laser sources to a target area.

17. The apparatus of claim 16, wherein the flexible waveguide includes an optical fiber with a core size of approximately 400 um.

18. The apparatus of claim 16, further comprising a handpiece, provided at a distal end of the flexible waveguide, configured to emit the laser light to the target area.

19. The apparatus of claim 18, wherein the flexible waveguide is provided with a pliable metal sheath surrounding the flexible waveguide and control wiring connected to the handpiece.

20. The apparatus of claim 16, wherein the plurality of laser sources comprise:
a first laser source to transmit laser light having a wavelength of approximately 660 nm at a power up to approximately 100 mW;
a second laser source to transmit laser light having a wavelength of approximately 800 nm in a power range of approximately 0.1 to 12.0 W;
a third laser source to transmit laser light having a wavelength of approximately 970 nm at a power range of approximately 0.1 to 12.0 W;
a fourth laser source to transmit laser light having a wavelength of approximately 905 nm at a power range of approximately 0.1 to 12.0 W.
